(19) United States
(12) Patent Application Publication
(10) Pub. No.: US 2025/0186523 A1
(43) Pub. Date: Jun. 12, 2025

(54) APPLICATION OF FRUITING BODY OF LYOPHYLLUM DECASTES IN PREPARING LYOPHYLLUM DECASTES FOOD OR LIPID-LOWERING DRUGS

(71) Applicants: Hexi University, Zhangye (CN); Lanzhou New District Qindong Agricultural Investment development Co., LTD, Lanzhou (CN)

(72) Inventors: Yali XI, Zhangye (CN); Shenglong WEI, Zhangye (CN); Xuecheng LI, Zhangye (CN); Zewen AN, Zhangye (CN); Jian ZHANG, Zhangye (CN); Xiaoming TIAN, Zhangye (CN); Shengyu GUO, Zhangye (CN); Qiannan CHAI, Zhangye (CN)

(73) Assignees: Hexi University, Zhangye (CN); Lanzhou New District Qindong Agricultural Investment development Co., LTD, Lanzhou (CN)

(21) Appl. No.: 18/963,834

(22) Filed: Nov. 29, 2024

(30) Foreign Application Priority Data
Dec. 8, 2023 (CN) 202311686854.5

(51) Int. Cl. A61K 36/07; A23L 31/00; A23P 10/22; A61P 3/06

(52) U.S. Cl. CPC A61K 36/07; A23L 31/00; A23P 10/22; A61P 3/06; A61K 2236/15; A61K 2236/331; A61K 2236/51

(57) ABSTRACT

An application of a fruiting body of Lyophyllum decastes in preparing Lyophyllum decastes food or lipid-lowering drugs is provided. The present disclosure takes the fruiting body of Lyophyllum decastes as only raw materials and adopts the ultrafine powder of the fruiting body of Lyophyllum decastes and the fungal polysaccharide concentrated solution of the fruiting body of Lyophyllum decastes to prepare Lyophyllum decastes food, which has the characteristics of unique composition, remarkable efficacy, safety and stability, and sufficient raw materials. Moreover, the products do not add any drugs, pigments, curing agents, flavoring agents, preservatives and other ingredients, with excellent lipid-lowering effects.

### CROSS REFERENCE TO THE RELATED APPLICATIONS

This application is based upon and claims priority to Chinese Patent Application No. 202311686854.5, filed on Dec. 8, 2023, the entire contents of which are incorporated herein by reference.

### TECHNICAL FIELD

The present disclosure relates to the technical field of biological products, and in particular, to an application of a fruiting body of Lyophyllum decastes in preparing Lyophyllum decastes food or lipid-lowering drugs.

### BACKGROUND

Overweight and obesity are important risk factors for a series of diseases such as diabetes, hyperlipidemia, hypertension, and snoring. Obesity not only affects physical beauty and brings inconvenience to life, but also increases the risk of cardiovascular disease and cancer, affects the function of the digestive system and endocrine system, reduces reproductive ability, and causes joint soft tissue injury, psychological disorder, heart disease, diabetes, atherosclerosis, fatty liver and other diseases. Obesity is the source of all diseases. Therefore, reasonable lipid-lowering has important practical significance for people's health and life.

### SUMMARY

An objective of the present disclosure is to provide an application of a fruiting body of Lyophyllum decastes in preparing Lyophyllum decastes food or lipid-lowering drugs, and polysaccharides from fruiting body of Lyophyllum decastes have excellent lipid-lowering effects.

In order to achieve the above objective, the present disclosure adopts the following technical solutions.

The present disclosure provides an application of a fruiting body of Lyophyllum decastes in preparing Lyophyllum decastes food, and a preparation method of Lyophyllum decastes food includes the following steps:

mixing ultrafine powder of the fruiting body of Lyophyllum decastes, fungal polysaccharide concentrated solution of fruiting body of Lyophyllum decastes, and sterile water, and performing granulation, polishing and drying in turn, to obtain Lyophyllum decastes food.

Preferably, a preparation method of the ultrafine powder of the fruiting body of Lyophyllum decastes includes the following steps: performing drying and ultrafine grinding on the fruiting body of Lyophyllum decastes in turn, to obtain the ultrafine powder of fruiting body of Lyophyllum decastes; and a diameter of the ultrafine powder of the fruiting body of Lyophyllum decastes is ≤0.5 m.

Preferably, a drying temperature is 50-55° C., and performing the drying until a water content of the fruiting body is 12-13 wt %.

Preferably, a mass concentration of fungal polysaccharide of fruiting body of Lyophyllum decastes in the fungal polysaccharide concentrated solution of fruiting body of Lyophyllum decastes is 68-70%; a dosage ratio of ultrafine powder of fruiting body of Lyophyllum decastes, fungal polysaccharide concentrated solution of fruiting body of Lyophyllum decastes and sterile water is 500 g:(400-450) mL:(40-50) mL.

Preferably, a preparation method of the fungal polysaccharide concentrated solution of the fruiting body of Lyophyllum decastes includes the following steps:
mixing fruiting body powder of Lyophyllum decastes and absolute alcohol to obtain a mixture, performing ultrasonic extraction on the mixture to obtain polyacetylene compounds, and segregating, to obtain filter residue;
mixing the filter residue and water to obtain a mixture, and performing ultrasonic extraction on the mixture to obtain polysaccharide, to obtain an extracting solution; and
performing vacuum concentration on the extracting solution, to obtain the fungal polysaccharide concentrated solution of the fruiting body of Lyophyllum decastes.

Preferably, a temperature of the ultrasonic extraction to obtain polyacetylene compounds is 55-60° C., a stirring speed is 35-40 r/min, ultrasonic frequency is 18-20 KHz, extraction times are 2 times, and time of each extraction is 2.5-3 h independently.

Preferably, a temperature of the ultrasonic extraction to obtain polysaccharide is 55-60° C., a stirring speed is 35-40 r/min, ultrasonic frequency is 18-20 KHz, extraction times are 2 times, and time of each extraction is 2.5-3 h.

Preferably, conditions of the vacuum concentration include: water bath temperature 85-90° C., rotating speed of an evaporator bottle 20 r/min, vacuum degree of decompression is 0.05-0.07 MPa, and temperature of condensation cycle temperature −15-−20° C.; and performing the vacuum concentration until a water content of the polysaccharide reaches 30-32 wt %.

Preferably, a drying temperature is 45-50° C., and performing the drying until a water content of materials is 12-13 wt %.

The present disclosure provides an application of a fruiting body of Lyophyllum decastes in preparing lipid-lowering drugs, and a preparation method of the lipid-lowering drugs includes the following steps:

mixing ultrafine powder of fruiting body of Lyophyllum decastes, fungal polysaccharide concentrated solution of fruiting body of Lyophyllum decastes, and sterile water, and performing granulation, polishing and drying in turn, to obtain the lipid-lowering drugs.

The present disclosure takes the fruiting body of Lyophyllum decastes as only raw materials and adopts the ultrafine powder of the fruiting body of Lyophyllum decastes and the fungal polysaccharide concentrated solution of the fruiting body of Lyophyllum decastes to prepare Lyophyllum decastes food, which has the characteristics of unique composition, remarkable efficacy, safety and stability, and sufficient raw materials. Moreover, the products do not add any drugs, pigments, curing agents, flavoring agents, preservatives and other ingredients, with excellent lipid-lowering effects.

Further, the present disclosure adopts ultrafine grinding, ultrasonic extraction of functional components, purification, vacuum concentration, granulation, polishing, and drying to produce Lyophyllum decastes food, wherein adopted technology including ultrafine grinding technology, ultrasonic extraction technology, vacuum evaporation concentration technology, etc. increases the content of fungal polysaccharide of the fruiting body of Lyophyllum decastes in the fungal polysaccharide concentrated solution of the fruiting body of Lyophyllum decastes, thereby effectively increasing the content (31-33 wt %) of fungal polysaccharide of fruiting body of Lyophyllum decastes in Lyophyllum decastes food or lipid-lowering drugs, ensuring the product quality, edible, preservation, easy to carry.

### DETAILED DESCRIPTION OF THE EMBODIMENTS

The present disclosure provides an application of a fruiting body of Lyophyllum decastes in preparing Lyophyllum decastes food, and a preparation method of Lyophyllum decastes food includes the following steps:

ultrafine powder of the fruiting body of Lyophyllum decastes, fungal polysaccharide concentrated solution of fruiting body of Lyophyllum decastes, and sterile water are mixed to obtain a mixture, and granulation, polishing and drying are performed on the mixture in turn, to obtain Lyophyllum decastes food.

In the present disclosure, a preparation method of the ultrafine powder of the fruiting body of Lyophyllum decastes includes the following steps:

drying and ultrafine grinding are performed on the fruiting body of Lyophyllum decastes in turn, to obtain the ultrafine powder of fruiting body of Lyophyllum decastes; and a diameter of the ultrafine powder of the fruiting body of Lyophyllum decastes is ≤0.5 m.

The present disclosure preferably adopts the fruiting body of Lyophyllum decastes with maturity of 70%-80%, no disease spot, no insect food and no impurity to prepare ultrafine powder.

In the present disclosure, a drying temperature is 50-55° C., and performing the drying until a water content of the fruiting body is 12-13 wt %; and drying is preferably performed in an electric blast drying oven.

In the present disclosure, ultrafine grinding is preferably performed by an ultrafine pulverizer.

In the present disclosure, a mass concentration of fungal polysaccharide of fruiting body of Lyophyllum decastes in the fungal polysaccharide concentrated solution of fruiting body of Lyophyllum decastes is preferably 68-70%; and a dosage ratio of ultrafine powder of fruiting body of Lyophyllum decastes, fungal polysaccharide concentrated solution of fruiting body of Lyophyllum decastes and sterile water is preferably 500 g:(400-450) mL:(40-50) mL, and more preferably 500 g:450 mL:50 mL.

In the present disclosure, a preparation method of the fungal polysaccharide concentrated solution of the fruiting body of Lyophyllum decastes preferably includes the following steps:
fruiting body powder of Lyophyllum decastes and absolute alcohol are mixed to obtain a mixture, ultrasonic extraction is performed on the mixture to obtain polyacetylene compounds, and then the polyacetylene compounds are separated, to obtain filter residue;
the filter residue and water are mixed to obtain a mixture, and ultrasonic extraction is performed on the mixture, to obtain an extracting solution of polysaccharide; and
vacuum concentration is performed on the extracting solution, to obtain the fungal polysaccharide concentrated solution of the fruiting body of Lyophyllum decast.

The present disclosure preferably adopts a Chinese medicine slicing grinder to grind the fruiting body that is dried until a water content of the fruiting body is 12-13 wt % into grinding powder with a particle size of 0.5-1 cm, to obtain the fruiting body powder of Lyophyllum decaste.

In the present disclosure, a mass ratio of the fruiting body powder of Lyophyllum decaste and absolute alcohol is preferably 1:10.

In the present disclosure, a temperature of the ultrasonic extraction to obtain polyacetylene compounds is preferably 55-60° C., a stirring speed is preferably 35-40 r/min, ultrasonic frequency is preferably 18-20 KHz, extraction times are preferably 2 times, and time of each extraction is preferably 2.5-3 h independently; a process of performing ultrasonic extraction to obtain polyacetylene compounds, and segregating, to obtain filter residue is preferably: the fruiting body powder of Lyophyllum decastes is put into an ultrasonic extraction tank, absolute alcohol is added to the ultrasonic extraction tank to obtain a mixture, the mixture is soaked for 10-12 h, a solvent temperature is controlled at 55-60° C., a stirring speed is controlled at 35-40 r/min, and ultrasonic frequency is controlled at 18-20 KHz, the fruiting body powder of Lyophyllum decastes is continuously extracted for 2.5-3 h, then a mixture obtained after extraction is filtered with 150-200-mesh filter screen, achieving the solid-liquid separation, to obtain the filtrate, thereby completing the first extraction; then absolute alcohol is added to the ultrasonic extraction tank for the second extraction, the extraction conditions are the same as before, and the filtrate and filter residue are obtained by filtration; and the obtained filter residue is dried in a dry-hot air blast drying box at 45-50° C. for the next extraction of polysaccharide of the fruiting body of Lyophyllum decastes.

In the present disclosure, a mass ratio of the filter residue to water is preferably 1:10, a temperature of the ultrasonic extraction to obtain polysaccharide is preferably 55-60° C., a stirring speed is preferably 35-40 r/min, ultrasonic frequency is preferably 18-20 KHz, extraction times are 2 times, and time of each extraction is 2.5-3 h.

In the present disclosure, a process of the ultrasonic extraction to obtain polysaccharide is preferably: the filter residue of the fruiting body of Lyophyllum decastes after extracting polyacetylene compounds is put into an ultrasonic extraction tank, drinking water is added to obtain a mixture, the filter residue is soaked for 10-12 h, a water temperature is controlled at 55-60° C., a stirring speed is controlled at 35-40 r/min, ultrasonic frequency is controlled at 18-20 KHz, the mixture is continuously extracted for 2.5-3 h; then a mixture obtained after extraction is filtered with 150-200-mesh filter screen, achieving the solid-liquid separation, to obtain filtrate, thereby completing the first extraction; and then drinking water is added to the ultrasonic extraction tank for the second extraction, the extraction conditions are the same as before, and the filtrate and filter residue are obtained by filtration; and two extracting solution are mixed to obtain an extracting solution.

In the present disclosure, conditions of the vacuum concentration preferably include: water bath temperature 85-90° C., rotating speed of an evaporator bottle 20 r/min, vacuum degree of decompression is 0.05-0.07 MPa, and temperature of condensation cycle temperature −15-−20° C.; and the vacuum concentration is performed until a water content of the polysaccharide reaches 30-32 wt %; the water bath temperature is more preferably 86-88° C., the vacuum degree of decompression is more preferably 0.06 MPa, and the temperature of condensation cycle temperature is more preferably −15-−18° C.; and the vacuum concentration is performed until a water content of the polysaccharide more preferably reaches 31 wt %.

In the present disclosure, ultrafine powder of the fruiting body of Lyophyllum decastes, fungal polysaccharide concentrated solution of fruiting body of Lyophyllum decastes, and sterile water are mixed to obtain a mixture, the mixture is added to a food agitator, the food agitator is turned on, the mixture is stirred at low speed (25-30 r/min) for 5 min, medium speed (35-40 r/min) for 3 min, and high speed (55-60 r/min) for 5 min until mixing uniformity; then products after stirring are added to a Chinese medicine granulator for granulating, the prepared pellets are put into a Chinese medicine polishing machine for polishing for 10 min, the polished pellets are placed in a dry-hot air blast drying box, and dried until a water content is 12-13 wt %, to obtain Lyophyllum decastes food; and a drying temperature is 45-50° C., and drying is performed until a water content of materials is preferably 12-13 wt %.

The present disclosure provides an application of a fruiting body of Lyophyllum decastes in preparing lipid-lowering drugs, and a preparation method of the lipid-lowering drugs includes the following steps:

ultrafine powder of fruiting body of Lyophyllum decastes, fungal polysaccharide concentrated solution of fruiting body of Lyophyllum decastes, and sterile water are mixed to obtain a mixture, and granulation, polishing and drying are performed on the mixture in turn, to obtain the lipid-lowering drugs.

A process for mixing ultrafine powder of the fruiting body of Lyophyllum decastes, fungal polysaccharide concentrated solution of fruiting body of Lyophyllum decastes, and sterile water, and the process of performing granulation, polishing and drying in turn is preferably the same as described above and will not be repeated here.

In the following, the technical solutions provided by the present disclosure are described in detail in combination with embodiments, but they cannot be understood as limiting the scope of protection of the present disclosure.

### Embodiment 1

Fresh, fruiting bodies of Lyophyllum decastes with maturity of 70%-80%, no disease spot, no insect food and no impurity were harvested, placed in a dry-hot air blast drying box at 55° C., maintained for 72 h, and dried to achieve a water content of 12% in the fruiting bodies, to obtain the fruiting bodies of Lyophyllum decastes;

the dried fruit bodies of Lyophyllum decastes were ground into 0.5 cm powder by a Chinese medicine slicing grinder, 1.5 kg of fruiting body powder of Lyophyllum decastes was weighed and put into an ultrasonic extraction tank, 15 kg of food-grade absolute alcohol was added at a mass ratio of 1:10, the fruiting body powder of Lyophyllum decastes was soaked for 12 h to obtain a mixture, a solvent temperature was set and controlled at 60° C., a stirring speed was set and controlled at 40 r/min, ultrasonic frequency was set and controlled at 20 KHz, the mixture was continuously extracted for 3 h, a mixture obtained after extraction was filtered with 200-mesh filter screen to obtain filtrate, thereby completing the first extraction; then 12 kg of absolute alcohol was added to the ultrasonic extraction tank for the second extraction, the extraction conditions were the same as before, and the filtrate and filter residue were obtained by filtration; the filter residue was dried in a dry-hot air blast drying box at 45° C. for the next extraction of polysaccharide of Lyophyllum decastes;

1.5 kg of the obtained filter residue was placed into the ultrasonic extraction tank, 15 kg of drinking water was added at a mass ratio of 1:10, the filter residue was soaked for 12 h to obtain a mixture, a solvent temperature was set and controlled at 60° C., a stirring speed was set and controlled at 40 r/min, ultrasonic frequency was set and controlled at 20 KHz, the mixture was continuously extracted for 3 h, a mixture obtained after extraction was filtered with 100-mesh filter screen to obtain filtrate, thereby completing the first extraction; then 12 kg of drinking water was added to the ultrasonic extraction tank for the second extraction, the extraction conditions were the same as before, and the filtrate and filter residue were obtained by filtration; and two extracting solution were mixed to obtain an extracting solution;

vacuum concentration was performed on the extracting solution with a rotary evaporator, a water bath temperature was set and controlled at 90° C., a rotating speed of the evaporation bottle was set and controlled at 20 r/min, vacuum degree of decompression was set and controlled at 0.07 MPa, a temperature of condensation cycle was set and controlled at −20° C., the above operation was performed until a water content of polysaccharide reached 32%, the polysaccharide concentrated solution of Lyophyllum decastes was obtained, and a concentration of the fungal polysaccharide of Lyophyllum decastes in the concentrated solution was 68%; and

the dried fruiting bodies of Lyophyllum decastes were ground into fine powder with particle diameter ≤0.5 micron by an ultrafine pulverizer, to obtain ultrafine powder of the fruiting bodies of Lyophyllum decastes, 500 g of the ultrafine powder of fruiting bodies of the Lyophyllum decastes, 450 mL of the polysaccharide concentrated solution of Lyophyllum decastes (derived from the extraction amount of 1500 g fruiting bodies), 50 mL of sterile water were weighed and added into a food agitator in turn to obtain a mixture, the food agitator was started, the mixture was stirred at low speed (30 r/min) for 5 min, medium speed (40 r/min) for 3 min, and high speed (60 r/min) for 5 min until evenly mixed to obtain products, the products were placed into a Chinese medicine slicing grinder to produce pellets, the prepared pellets were put into a Chinese medicine polishing machine for polishing for 10 min, and the polished pellets were placed in a dry-hot air blast drying box at 50° C. for 72 h and dried until a water content was 12% and single pellet weight was 0.085 g, to obtain Lyophyllum decastes food or lipid-lowering drugs (a content of fungal polysaccharide in the fruiting bodies of Lyophyllum decastes was 33 wt %), i.e. the “lipid-lowering pills” described below.

### Experimental Example of Lipid-Lowering Effect

### 1. Methods and Materials

### 1) Experimental Animals

SD rats, SPF grade, weight 200-240 g, male, 30 rats; and feed in an SPF-grade animal room, room temperature 24±2° C., relative humidity 40-70%.

### 2) Model Diets

Components of model diets for rats: lard 5%, casein 5%, cholesterol 1.2%, sodium cholate 0.2%, calcium hydrogen phosphate 0.6%, stone powder 0.4%, and maintenance diets 87.6%.

Except for crude fat, other quality indicators of the model diets should reach the national standards for maintenance diets.

The model diets were produced by Beijing Huafukang Biotechnology Co., Ltd., License No.: SCXK (Beijing) 2019-0008; and

the maintenance diets came from Beijing Huafukang Biotechnology Co., Ltd.

### 3) Instruments and Reagents

Total cholesterol in serum (TC) assay kits, BioSino Bio-Technology and Science Inc.; triglyceride (TG) assay kits, BioSino Bio-Technology and Science Inc.; low-density lipoprotein cholesterol (LDL-C) assay kits, BioSino Bio-Technology and Science Inc.; high-density lipoprotein cholesterol (HDL-C) assay kits, BioSino Bio-Technology and Science Inc.; HITACHI 3100 automatic biochemical analyzer, Hitachi Diagnostic Products (Shanghai) Co., Ltd.; TDL-5-A low-speed table centrifuge, Shanghai Anting Scientific Instrument Factory; electronic analytical balance, Mettler-Toledo Instrument (Shanghai) Ltd.; and MP-C electronic balance, Shanghai Sunny Hengping Instrument Ltd.

### 4) Dose Group Classification and Treatment Time of Test Samples

The experiment was set up with three treatments, namely: 1 dose group of test substance (lipid-lowering pills 3 g/kg), 1 blank control group and 1 model control group. The treatment time of test samples was 28 d. The grouping situation is shown in Table 1.

TABLE 1
Grouping situation

| Group | Test substance | Feed | Quantity |
|---|---|---|---|
| 1 | Blank control group | Normal blank | 10 |
| | (distilled water) | rats + maintenance diets | |
| 2 | Model control group | Model rats + model diets | 10 |
| | (distilled water) | | |
| 3 | Dose group | Model rats + model | 10 |
| | (lipid-lowering pills | diets + lipid-lowering | |
| | 3 g/kg) | pills | |

### 2. Experimental Procedures

1) Adaptation period: animals were fed with maintenance diets and observed for 5-7 d under a barrier system.

2) Model establishment period

Rats were randomly divided into two groups according to body weight. Ten animals were given maintenance diets as a blank control group, and 20 animals were given model diets as a model group (model control group and dose group). After the model group was given model diets for 6 weeks, the rats in the blank control group and the model group were subjected to blood collection without fasting (inner canthus or tail), the serum was separated as soon as possible after blood collection, and the levels of serum TC, TG, LDL-C and HDL-C were measured. According to the level of TC, the model group was randomly divided into 2 groups (1 model control group and 1 dose group), then, the model control group and dose group were compared, their values of TC, TG, LDL-C, and HDL-C had no significant differences (P>0.05); and the model control group and the dose group were compared with the blank control group, respectively, their values of TC, TG and LDL-C were all increased and had significant differences (P<0.05), and it was determined that the model was established.

3) Feeding of test samples

After grouping, the dose group (lipid-lowering pill 3 g/kg) was fed with the test samples by gastric irrigation daily, the blank control group and the model control group were fed with the same volume of distilled water simultaneously, the blank control group continued to be fed with maintenance diets, the model control group and the dose group continued to be fed with maintenance diets, rats of three groups were weighed regularly and were subjected to blood collection without fasting, the serum was separated as soon as possible after blood collection, and the levels of serum TC, TG, LDL-C and HDL-C were measured.

4) Observed indexes: Total cholesterol in serum (TC), triglyceride (TG), low-density lipoprotein cholesterol (LDL-C), and high-density lipoprotein cholesterol (HDL-C).

5) Data processing and result determination

Variance analysis was adopted, firstly, variance homogeneity test was performed according to the procedure of variance analysis, to calculate an F value, when the F value was <F0.05, conclusion: there was no significant difference among the mean values of each group; when the F value was ≥F0.05 and P was ≤0.05, the pairwise comparison method of means between multiple experimental groups and one control group was used for statistics; appropriate variable conversion was performed on non-normal or unequal-variance data, and after meeting the requirements of normality or homogeneity of variance, the converted data were used for statistics; and if the variable had not yet reached the purpose of normality or homogeneity of variance after conversion, it could be changed to rank and test for statistics.

6) Result determination of animal experiments

The result determination which was helpful to maintain health level function of the serum cholesterin: the model control group and blank control group were compared, total cholesterol in serum or low-density lipoprotein cholesterol increased, the differences were significant, there was no significant difference in serum triglyceride, and it was determined that the model was established. Each dose group and model control group were compared, total cholesterol in serum or low-density lipoprotein cholesterol decreased in any dose group, differences were significant, and the high-density lipoprotein cholesterol in serum of each dose group was not significantly lower than that in the model control group, serum triglyceride was not significantly higher than that in the model control group, it could be determined that the test sample was helpful to maintain the health level of blood cholesterol and the results of animal experiments were positive.

The results indicated that:

1) The results of the effect on body weight of hypercholesterolemia model rats are shown in Table 2.

TABLE 2
Effect on body weight of hypercholesterolemia model rats (x̅ ± s and n = 10)

| | | | | | | Weight gain |
|---|---|---|---|---|---|---|
| | | | | | | at the end |
| | | | | | | of the |
| | Body weight (g) | | | | | experiment |
| Group | 0 W | 1 W | 2 W | 3 W | 4 W | (g) |
| Blank control | 536.16 ± | 554.11 ± | 576.55 ± | 590.07 ± | 602.99 ± | 66.83 ± |
| | 32.84^{#} | 37.05 | 39.37 | 41.54 | 44.23 | 15.88 |
| Model control | 585.51 ± | 602.69 ± | 621.78 ± | 634.79 ± | 655.75 ± | 70.24 ± |
| | 41.65 | 51.15 | 53.68 | 52.82 | 52.73 | 18.07 |
| Dose group | 541.09 ± | 559.81 ± | 573.31 ± | 585.97 ± | 597.70 ± | 56.61 ± |
| (lipid-lowering | 53.89 | 57.80 | 52.43 | 45.47 | 50.92^{#} | 20.04 |
| pills) | | | | | | |

Note:
Compared with the model group,
^{#}P < 0.05.

It could be seen from Table 1 that before administration, compared with the blank control group, the body weight of the model control group was significantly increased; and compared with the model control group, the body weight of the rats in the model control group increased at the same time point after administration, but there was no statistical difference. At the same time point of 0-3 weeks, there was no significant difference in body weight between the dose group (3 g/kg) and the model control group; and at the 4th week of the experiment, compared with the model control group, the body weight of rats in the dose group (3 g/kg) was significantly decreased (P<0.05).

2) The effect on blood lipid TC, TG, HDL-C, LDL-C in hypercholesterolemia model rats is shown in Table 3-5.

TABLE 3
Changes of TC, TG, HDL-C and LDL-C in hypercholesterolemia
model rats before feeding the test samples
(X̅ ± S and n = 10)

| | TC | TG | HDL-C | LDL-C |
|---|---|---|---|---|
| Group | mmol/L | mmol/L | mmol/L | mmol/L |
| Blank | 1.91 ± 0.16^{##} | 1.47 ± 0.38^{##} | 0.81 ± 0.07 | 0.17 ± 0.03^{##} |
| control | | | | |
| Model | 2.61 ± 0.22 | 2.10 ± 0.41 | 0.88 ± 0.09 | 0.50 ± 0.09 |
| control | | | | |
| Dose | 2.59 ± 0.16 | 2.03 ± 0.51 | 0.85 ± 0.11 | 0.53 ± 0.13 |
| group | | | | |
| (lipid- | | | | |
| lower- | | | | |
| ing | | | | |
| pills) | | | | |

Note:
Compared with the model group,
^{#}P < 0.05.

It could be seen from Table 3 that before feeding the test samples: compared with the blank control group, the TC, TG and LDL-C of the model control group were all highly significantly increased (P<0.01), and there was no significant difference in HDL-C. Compared with the model control group, there was no significant difference in TC, TG, LDL-C and HDL-C in the dose group (3 g/kg).

TABLE 4
Changes of TC, TG, HDL-C and LDL-C in hypercholesterolemia
model rats fed with the test samples for 15 d

| | TC | TG | HDL-C | LDL-C |
|---|---|---|---|---|
| Group | mmol/L | mmol/L | mmol/L | mmol/L |
| Blank | 2.04 ± 0.18^{##} | 1.58 ± 0.49^{#} | 0.85 ± 0.08 | 0.19 ± 0.03^{##} |
| control | | | | |
| Model | 2.88 ± 0.23 | 2.31 ± 0.69 | 0.89 ± 0.07 | 0.54 ± 0.12 |
| control | | | | |
| Dose | 2.71 ± 0.24 | 1.87 ± 0.55 | 0.84 ± 0.10 | 0.55 ± 0.16 |
| group | | | | |
| (lipid- | | | | |
| lower- | | | | |
| ing | | | | |
| pills) | | | | |

Note:
Compared with the model group,
^{#}P < 0.05 and
^{##}P < 0.01.

It could be seen from Table 4 that when the test samples were fed for 15 d: compared with the blank control group, the levels of TC, TG and LDL-C in the model control group were all significantly or highly significantly increased (P<0.05 and P<0.01), but there was no significant difference in HDL-C. Compared with the model control group, TC, TG, LDL-C and HDL-C in the dose group (3 g/kg) were not significantly decreased.

TABLE 5
Changes of blood lipid TC, TG, HDL-C and
LDL-C in hypercholesterolemia model rats
after 28 d of feeding the test samples.

| | TC | TG | HDL-C | LDL-C |
|---|---|---|---|---|
| Group | mmol/L | mmol/L | mmol/L | mmol/L |
| Blank | 2.05 ± 0.18^{##} | 1.65 ± 0.61^{##} | 0.88 ± 0.06 | 0.17 ± 0.04^{##} |
| control | | | | |
| Model | 2.99 ± 0.23 | 2.54 ± 0.67 | 0.90 ± 0.12 | 0.64 ± 0.11 |
| control | | | | |
| Dose | 2.60 ± 0.23^{##} | 1.66 ± 0.48^{##} | 0.88 ± 0.11 | 0.56 ± 0.17 |
| group | | | | |
| (lipid- | | | | |
| lower- | | | | |
| ing | | | | |
| pills) | | | | |

Note:
Compared with the model group,
^{##}P < 0.01.

It could be seen from Table 5 that when the test sample was fed for 28 d, compared with the blank control group, the levels of TC, TG and LDL-C in the model control group were increased, with very significant differences, and there was no significant difference in HDL-C. Compared with the model control group, the blood lipid TC and TG in the dose group (3 g/kg) were significantly decreased, and the differences were significant (P<0.01); and the levels of LDL-C and HDL-C were decreased, and there was no significant difference.

It could be seen from the above embodiments:

1) At the 4th week of the experiment, compared with the model control group, the body weight of the rats in the dose group (3 g/kg) was significantly decreased (P<0.05); and it indicated that the dose group (3 g/kg) had a significant reduction effect on body weight of hypercholesterolemia model rats.

2) Before feeding the test sample and feeding the test sample for 15 d: compared with the model control group, blood lipid TC, TG, LDL-C and HDL-C of hypercholesterolemia model rats in the dose group (3 g/kg) were not significantly decreased.

3) Feeding the test samples for 28 d: compared with the blank control group, the levels of TC, TG and LDL-C in the model control group were all increased, with very significant differences, and there was no significant difference in HDL-C between the two groups. Compared with the model control group, the dose group (3 g/kg) significantly decreased TC and TG in hypercholesterolemia model rats, with significant differences (P<0.01), and LDL-C and HDL-C in serum were not significantly lower than those in the model control group.

The above results show that the test samples “lipid-lowering pills” prepared by the present disclosure have a significant effect on reducing blood lipids in rats, which are an ideal product for preventing and decreasing blood lipids.

The above descriptions are only the preferred embodiments of the present disclosure. It is to be pointed out that those of ordinary skill in the art can also make several improvements and modifications without departing from the principle of the present disclosure, and such improvements and modifications shall fall within the protection scope of the present disclosure.

What is claimed is:

1. A method of preparing a Lyophyllum decastes food using a fruiting body of Lyophyllum decastes, comprising the following steps:
mixing an ultrafine powder of the fruiting body of the Lyophyllum decastes, a fungal polysaccharide concentrated solution of the fruiting body of the Lyophyllum decastes, and sterile water, and performing a granulation, a polishing, and a first drying in turn, to obtain the Lyophyllum decastes food;
wherein a preparation method of the ultrafine powder of the fruiting body of the Lyophyllum decastes comprises the following steps: performing a second drying and an ultrafine grinding on the fruiting body of the Lyophyllum decastes in turn, to obtain the ultrafine powder of the fruiting body of the Lyophyllum decastes; and wherein a diameter of the ultrafine powder of the fruiting body of the Lyophyllum decastes is ≤0.5 m;
a preparation method of the fungal polysaccharide concentrated solution of the fruiting body of the Lyophyllum decastes comprises the following steps:
mixing a fruiting body powder of the Lyophyllum decastes and absolute alcohol to obtain a first mixture, performing an ultrasonic extraction on the first mixture to obtain polyacetylene compounds, and segregating, to obtain a filter residue;
mixing the filter residue and water to obtain a second mixture, and performing an ultrasonic extraction on the second mixture to obtain a fungal polysaccharide of the fruiting body of the Lyophyllum decastes, to obtain an extracting solution;
performing a vacuum concentration on the extracting solution, to obtain the fungal polysaccharide concentrated solution of the fruiting body of the Lyophyllum decastes;
wherein a mass concentration of the fungal polysaccharide of the fruiting body of the Lyophyllum decastes in the fungal polysaccharide concentrated solution of the fruiting body of the Lyophyllum decastes is 68-70%;
a temperature of the ultrasonic extraction to obtain the polyacetylene compounds is 55-60° C., a stirring speed of the ultrasonic extraction to obtain the polyacetylene compounds is 35-40 r/min, an ultrasonic frequency of the ultrasonic extraction to obtain the polyacetylene compounds is 18-20 KHz, extraction times of the ultrasonic extraction to obtain the polyacetylene compounds are 2 times, and a time of each ultrasonic extraction to obtain the polyacetylene compounds is 2.5-3 h independently;
a temperature of the ultrasonic extraction to obtain the fungal polysaccharide of the fruiting body of the Lyophyllum decastes is 55-60° C., a stirring speed of the ultrasonic extraction to obtain the fungal polysaccharide of the fruiting body of the Lyophyllum decastes is 35-40 r/min, an ultrasonic frequency of the ultrasonic extraction to obtain the fungal polysaccharide of the fruiting body of the Lyophyllum decastes is 18-20 KHz, extraction times of the ultrasonic extraction to obtain the fungal polysaccharide of the fruiting body of the Lyophyllum decastes are 2 times, and a time of each ultrasonic extraction to obtain the fungal polysaccharide of the fruiting body of the Lyophyllum decastes is 2.5-3 h;
a mass ratio of the fruiting body powder of the Lyophyllum decastes to the absolute alcohol is 1:10; and
a mass ratio of the filter residue to the water is 1:10.

2. The method according to claim 1, wherein a drying temperature of the second drying is 50-55° C., and performing the second drying until a water content of the fruiting body of the Lyophyllum decastes is 12-13 wt %.

3. The method according to claim 1, wherein a dosage ratio of the ultrafine powder of the fruiting body of the Lyophyllum decastes, the fungal polysaccharide concentrated solution of the fruiting body of the Lyophyllum decastes, and the sterile water is 500 g:(400-450) mL:(40-50) mL.

4. The method according to claim 1, wherein conditions of the vacuum concentration comprise: a water bath temperature of 85-90° C., a rotating speed of an evaporator bottle of 20 r/min, a vacuum degree of decompression of 0.05-0.07 MPa, and a condensation cycle temperature of −15-−20° C.; and performing the vacuum concentration until a water content of the fungal polysaccharide of the fruiting body of the Lyophyllum decastes reaches 30-32 wt %.

5. The method according to claim 1, wherein a drying temperature of the first drying is 45-50° C., and performing the first drying until a water content of materials is 12-13 wt %.

6. A method of preparing lipid-lowering drugs using a fruiting body of Lyophyllum decastes, comprising the following steps:
mixing an ultrafine powder of the fruiting body of the Lyophyllum decastes, a fungal polysaccharide concentrated solution of the fruiting body of the Lyophyllum decastes, and sterile water, and performing a granulation, a polishing, and a drying in turn, to obtain the lipid-lowering drugs.
